(19) United States
(12) Patent Application Publication
(10) Pub. No.: US 2026/0014550 A1
(43) Pub. Date: Jan. 15, 2026

(54) CATALYST STRUCTURE, FIXED-BED REACTOR, AND METHOD OF MANUFACTURING CATALYST STRUCTURE

(71) Applicant: IHI Corporation, Tokyo (JP)

(72) Inventor: Hiroyuki KAMATA, Tokyo (JP)

(73) Assignee: IHI Corporation, Tokyo (JP)

(21) Appl. No.: 19/338,613

(22) Filed: Sep. 24, 2025

Related U.S. Application Data
(63) Continuation of application No. PCT/JP2024/011832, filed on Mar. 26, 2024.

(30) Foreign Application Priority Data
May 30, 2023 (JP) 2023-088819

(51) Int. Cl. B01J 27/22; B01J 23/745; B01J 37/00; B01J 37/02; B01J 37/16; C07C 1/12

(52) U.S. Cl. CPC B01J 27/22; B01J 23/745; B01J 37/0009; B01J 37/0201; B01J 37/16; C07C 1/12

(57) ABSTRACT

A catalyst structure includes: an iron-based catalyst containing an Fe₅C₂ phase, and at least one of an Fe₂O₃ phase or an Fe₃O₄ phase; and a porous carrier for supporting the iron-based catalyst. The catalyst structure configures to synthesize hydrocarbons containing lower olefins. A fixed-bed reactor includes: the catalyst structure; and a reaction tube which contains the catalyst structure.

### CROSS REFERENCE TO RELATED APPLICATIONS

This application is a continuation application of International Application No. PCT/JP2024/011832, filed on Mar. 26, 2024, which claims priority to Japanese Patent Application No. 2023-088819, filed on May 30, 2023, the entire contents of which are incorporated by reference herein.

### BACKGROUND

### 1. Technical Field

This disclosure relates to a catalyst structure, a fixed-bed reactor, and a method of a manufacturing catalyst structure.

### 2. Description of the Related Art

Conventionally, a method of synthesizing hydrocarbons by the FT reaction (Fischer-Tropsch reaction) is known. The FT reaction progresses as shown in the following Chemical equation (1), and an iron-based catalyst and a cobalt-based catalyst are used as catalysts.

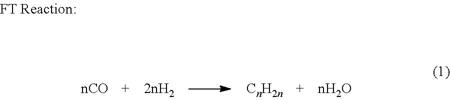

WO 2014/142282A1 discloses a catalyst for producing light hydrocarbons from a synthesis gas. The catalyst contains a metal-based compound active in the FT synthesis reaction, a catalyst for producing hydrocarbons from a synthesis gas, and a catalyst for decomposing the produced hydrocarbons to reduce their weight. WO 2014/142282A1 discloses that the metal-based compound contains cobalt.

### SUMMARY

However, carbon dioxide is regarded as a cause of global warming, and an attempt has been made to synthesize hydrocarbons containing lower olefins from carbon dioxide. The reaction to synthesize hydrocarbons from carbon dioxide progresses in two steps as shown in the following Chemical equations (2) and (1), and an iron-based catalyst is used.

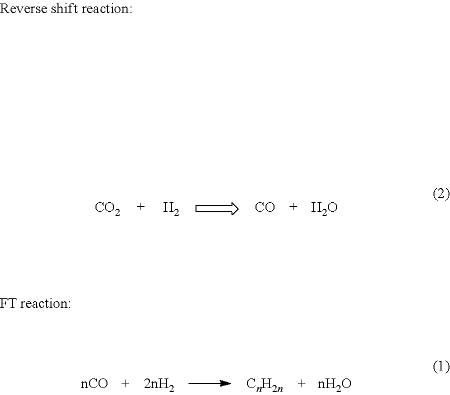

The active species of Chemical equation (2) is Fe₂O₃ phase (hematite phase) or Fe₃O₄ phase (magnetite phase), and carbon monoxide is produced from a raw material containing carbon dioxide and hydrogen by Chemical equation (2). The active species of Chemical equation (1) is Fe₅C₂ phase called Hagg carbide, and hydrocarbons containing lower olefins are produced by Chemical equation (1). Therefore, when the iron-based catalyst contains Fe₅C₂ phase, and at least one of an Fe₂O₃ phase or an Fe₃O₄ phase, hydrocarbons can be produced from a raw material containing carbon dioxide and hydrogen.

However, when an iron-based catalyst as described above is molded into pellets by a method such as extrusion molding, there is a possibility that Fe₂O₃ or Fe₃O₄ in the catalyst reacts with CO in the gas phase to form carbide components, and catalyst pellets are pulverized. Therefore, when the catalyst pellets are filled in a fixed-bed reactor and used, there is a possibility that the catalyst pellets are pulverized, reducing voids in the reaction tube, causing the pressure loss during the reaction to increase, and in the worst case, causing blockage in the reaction tube. In such a case, it can be difficult to remove the pulverized catalyst from the reaction tube, and the maintenance of the catalyst exchange may become difficult. Such pulverization of the catalyst pellets has not been confirmed for cobalt-based catalysts. It is a problem unique to iron-based catalysts.

Accordingly, it is an object of the present disclosure to provide a catalyst structure, a fixed-bed reactor, and a method of manufacturing the catalyst structure capable of mitigating pulverization.

A catalyst structure of the present disclosure includes: an iron-based catalyst containing an Fe₅C₂ phase, and at least one of an Fe₂O₃ phase or an Fe₃O₄ phase; and a porous carrier for supporting the iron-based catalyst. The catalyst structure configures to synthesize hydrocarbons containing lower olefins.

A volume of the catalyst structure may be 0.5 mm³ or more, and 1000 mm³ or less.

The porous carrier may be formed of metal.

Porosity of the porous carrier may be 85% to 95%.

A fixed-bed reactor according to the present disclosure includes a plurality of catalyst structures; and a reaction tube which contains the plurality of catalyst structures.

Hydrocarbons may be synthesized from raw a material containing carbon dioxide and hydrogen.

Hydrocarbons may be synthesized from a raw material containing carbon monoxide, carbon dioxide, and hydrogen.

A method of manufacturing a catalyst structure according to the present disclosure includes supporting an iron-based catalyst precursor with a porous carrier by impregnating a slurry containing a binder and the iron-based catalyst precursor into a porous carrier, and drying and calcining the slurry. The method of manufacturing the catalyst structure includes reducing the iron-based catalyst precursor supported with the porous carrier. The method of manufacturing a catalyst structure includes activating a reduced iron-based catalyst precursor with an activation gas containing at least one of carbon monoxide or carbon dioxide to produce an iron-based catalyst containing an Fe₅C₂ phase, and at least one of an Fe₂O₃ phase or an Fe₃O₄ phase. The catalyst structure includes the iron-based catalyst and the porous carrier for supporting the iron-based catalyst, and configures to synthesize hydrocarbons containing lower olefins.

The binder may contain at least one of an alumina binder or a silica binder.

According to the present disclosure, a catalyst structure, a fixed-bed reactor, and a method of manufacturing a catalyst structure capable of mitigating pulverization can be provided.

### BRIEF DESCRIPTION OF THE DRAWINGS

FIG. 1 is a schematic diagram illustrating a catalyst structure according to an embodiment.

FIG. 2 is a schematic diagram illustrating a catalyst structure according to an embodiment.

FIG. 3 is a schematic diagram illustrating a reaction system used in Examples and Comparative Examples.

FIG. 4 is a photograph showing an appearance of a catalyst structure of Example 1 before reaction and after activation when the catalyst structure is reacted under various conditions.

FIG. 5 is a photograph showing an appearance of a catalyst structure of Comparative Example 1 before reaction and after activation when the catalyst structure is reacted under various conditions.

FIG. 6 is a photograph showing an appearance of a catalyst structure of Comparative Example 2 before reaction and after activation when the catalyst structure is reacted under various conditions.

FIG. 7 is a graph illustrating a relationship between activation time and pressure loss when the catalyst structure of Example 1 is reacted under the conditions of Run 4.

FIG. 8 is a graph illustrating a relationship between activation time and pressure loss when the catalyst structure of Comparative Example 1 is reacted under the conditions of Run 4.

FIG. 9 is a graph illustrating a relationship between activation time and pressure loss when the catalyst structure of Comparative Example 2 is reacted under the conditions of Run 4.

FIG. 10 is an X-ray diffraction pattern of an iron-based catalyst after reaction of the catalyst structure of Example 1 under the conditions of Run 0.

FIG. 11 is an X-ray diffraction pattern of an iron-based catalyst after reaction of the catalyst structure of Example 1 under the conditions of Run 1.

FIG. 12 is an X-ray diffraction pattern of an iron-based catalyst after reaction of the catalyst structure of Example 1 under the conditions of Run 2.

FIG. 13 is an X-ray diffraction pattern of an iron-based catalyst after reaction of the catalyst structure of Example 1 under the conditions of Run 3.

FIG. 14 is an X-ray diffraction pattern of an iron-based catalyst after reaction of the catalyst structure of Example 1 under the conditions of Run 4.

### DESCRIPTION OF THE EMBODIMENTS

Several exemplary embodiments will be described below with reference to the drawings. The dimensional ratios in the drawings are exaggerated for the sake of explanation, and may differ from the actual ratios.

### [Catalyst Structure]

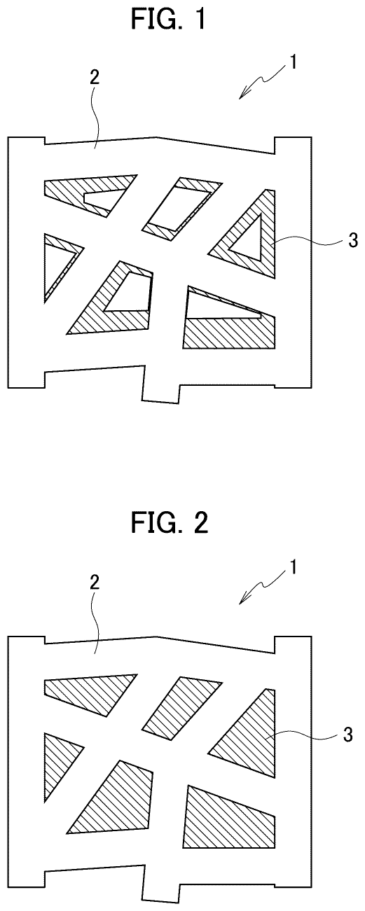

First, a catalyst structure 1 according to this embodiment will be described. As illustrated in FIG. 1, the catalyst structure 1 according to this embodiment includes an iron-based catalyst 2 and a porous carrier 3.

The iron-based catalyst 2 includes an Fe₅C₂ phase and at least one of an Fe₂O₃ phase or an Fe₃O₄ phase. When the iron-based catalyst 2 includes an Fe₅C₂ phase, an FT reaction represented by the following Chemical equation (1) can proceed. Further, when the iron-based catalyst 2 includes at least one of an Fe₂O₃ phase or an Fe₃O₄ phase, a reverse shift reaction represented by the following Chemical equation (2) can proceed. That is, when the iron-based catalyst 2 includes an Fe₅C₂ phase, and at least one of an Fe₂O₃ phase or an Fe₃O₄ phase, the following Chemical equations (1) and (2) can proceed to synthesize hydrocarbons containing lower olefins from a raw material containing carbon dioxide and hydrogen.

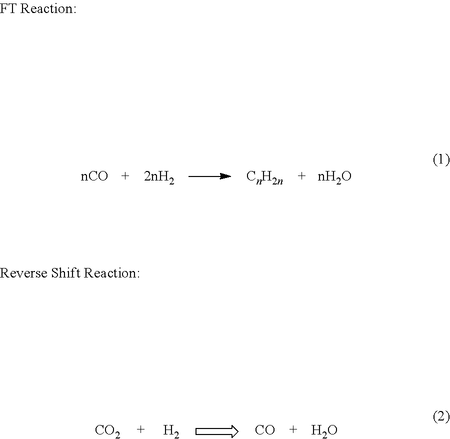

Here, Fe₂O₃ or Fe₃O₄ of the iron-based catalyst 2 reacts with CO in the gas phase depending on the reaction conditions, and a reaction to generate carbide components progresses with time. As Fe₂O₃ or Fe₃O₄ changes to carbide components, the density and volume of the iron-based catalyst 2 change from a microscopic viewpoint. In other words, iron oxide in catalyst pellets carbonizes, and pulverization of the pellets progresses. This tendency is especially remarkable at a high temperature. For example, when the temperature is higher than 300° C., the phase change to carbide progresses strongly, and excess carbon precipitates, eventually causing catalyst pellet pulverization.

Therefore, the porous carrier 3 supports the iron-based catalyst 2. When the iron-based catalyst 2 is molded into pellets by a method such as extrusion molding, the catalyst pellets may pulverize during use. However, the pulverization can be mitigated by supporting the iron-based catalyst 2 with the porous carrier 3. Thus, pressure loss in a reaction tube can be reduced, and blockage in the reaction tube caused by a pulverized catalyst can be mitigated. Moreover, the pulverization of the iron-based catalyst 2 can be mitigated, so that replacement of the catalyst structure 1 in the reaction tube can be facilitated.

The porous carrier 3 has an open cell type structure, and includes interconnected pores or through pores. That is, the porous carrier 3 includes open pores. As illustrated in FIGS. 1 and 2, the porous carrier 3 may include a network structure. As illustrated in FIG. 1, the iron-based catalyst 2 covers a solid surface forming pores inside the porous carrier 3, and the catalyst structure 1 may include pores therein. As illustrated in FIG. 2, the iron-based catalyst 2 is filled inside the porous carrier 3, and the catalyst structure 1 does not need to substantially include pores therein. A support amount of the iron-based catalyst 2 may be 5% by weight to 45% by weight, 10% by weight to 40% by weight, 15% by weight to 35% by weight, or 20% by weight to 30% by weight. The support amount of the iron-based catalyst 2 is a rate of the weight of the iron-based catalyst 2 to the weight of an entire catalyst structure 1.

The porous carrier 3 may include at least one material selected from the group consisting of metals, ceramics, and silicon carbide (SiC). For example, when the porous carrier 3 includes metal, heat conduction in the catalyst structure 1 can be promoted. Thus, the temperature distribution of the catalyst structure 1 becomes uniform, and generation of locally high-temperature hot spots can be mitigated. Therefore, an excessive phase transition of iron components of the iron-based catalyst 2 to carbide, and deposition of carbon due to hot spots can be mitigated. Specifically, the porous carrier 3 may be an Ni alloy or a Fe alloy. More specifically, the porous carrier 3 may be an NiFeCr alloy. The porous carrier 3 may be a FeCrAl alloy.

The porosity of the porous carrier 3 may be 85% to 95%. The pressure loss can be further mitigated when the porosity of the porous carrier 3 is 85% or more. A mechanical strength of the porous carrier 3 can be further improved when the porosity of the porous carrier 3 is 95% or less. The porosity can be calculated by dividing the volume of pores by the volume of an entire porous carrier 3.

An average pore diameter of the porous carrier 3 may be 500 μm to 4000 μm. The pressure loss can be further reduced when the average pore diameter of the porous carrier 3 is 500 μm or more. The mechanical strength of the porous carrier 3 can be further improved when an average pore diameter of the porous carrier 3 is 4000 μm or less. The average pore diameter can be obtained by measuring the number of cells per predetermined length of the porous carrier 3 and calculating an inverse of a measured value.

The catalyst structure 1 synthesizes hydrocarbons containing lower olefins. The lower olefins may contain at least any hydrocarbon having 2 to 4 carbon atoms. The lower olefins may contain, for example, at least one hydrocarbon selected from the group consisting of ethylene, propylene, 1-butene, 2-butene, and isobutene. The lower olefins can be used, for example, as raw materials for plastics. It should be noted that the catalyst structure 1 may synthesize hydrocarbons other than the lower olefins in addition to the lower olefins.

The catalyst structure 1 may be pellets. With the catalyst structure 1 in the form of pellets, a gas from a reaction raw material diffuses and improves heat conduction when the pellets of a plurality of the catalyst structures 1 are filled in a reaction tube. The shape of the catalyst structure 1 includes, but is not particularly limited to, cube, rectangular parallelepiped, cylindrical, spherical, ellipsoidal, or an unstable shape. The size of the catalyst structure 1 may be 1 mm to 10 mm, 1.5 mm to 7 mm, or 2 mm to 5 mm. The size of the catalyst structure 1 is a value of a length of one side calculated from the volume of the catalyst structure 1, assuming that the catalyst structure 1 is a cube.

The volume of the catalyst structure 1 may be 0.5 mm³ or more, and 1000 mm³ or less. When the volume of the catalyst structure 1 is 0.5 mm³ or more, it is possible to control the catalyst structure 1 to not flow out of the reaction tube during the reaction. In addition, when the volume of the catalyst structure 1 is 30 cm³ or less, it is possible to improve the ability to fill the reaction tube. The volume of the catalyst structure 1 may be 5 mm³ or more, 10 mm³ or more, and 15 mm³ or more. The volume of the catalyst structure 1 may be 500 mm³ or less, 200 mm³ or less, and 50 mm³ or less.

As described above, the catalyst structure 1 according to this embodiment includes the iron-based catalyst 2 containing an Fe₅C₂ phase, and at least one of an Fe₂O₃ phase or an Fe₃O₄ phase; and the porous carrier 3 for supporting the iron-based catalyst 2. The catalyst structure 1 synthesizes hydrocarbons containing lower olefins.

In the catalyst structure 1 according to this embodiment, the porous carrier 3 supports the iron-based catalyst 2, so that it is possible to mitigate pulverization compared with a case where the extrusion-molded catalyst pellets are used. Therefore, it is possible to mitigate the increase of pressure loss during the reaction and to facilitate maintenance of catalyst exchange by using the catalyst structure 1 according to this embodiment.

### [Fixed-Bed Reactor]

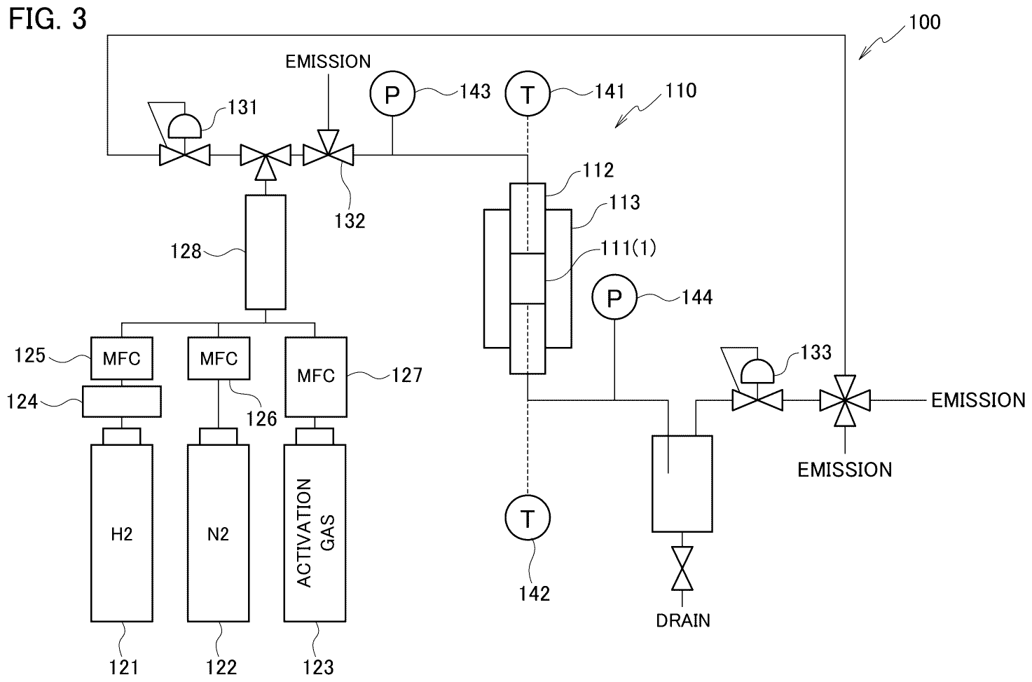

Next, a fixed-bed reactor 110 according to this embodiment will be described with reference to FIG. 3. As illustrated in FIG. 3, the fixed-bed reactor 110 according to this embodiment includes the catalyst structure 1, and a reaction tube 112 which contains the catalyst structure 1. The catalyst structure 1 is the same as the catalyst structure 1 described above, and the reaction tube 112 is filled with a plurality of the catalyst structures 1. The fixed-bed reactor 110 may include an electric furnace 113, and the reaction tube 112 may be installed in the electric furnace 113.

As described above, the catalyst structure 1 includes the iron-based catalyst 2 containing an Fe₅C₂ phase, and at least one of an Fe₂O₃ phase or an Fe₃O₄ phase, and synthesizes hydrocarbons containing lower olefins. Therefore, the fixed-bed reactor 110 can synthesize hydrocarbons containing lower olefins from raw materials containing carbon dioxide and hydrogen. By synthesizing hydrocarbons containing lower olefins from carbon dioxide, emission of carbon dioxide, which contributes to global warming, into the atmosphere can be reduced and effectively utilized. The raw materials may further contain carbon monoxide. In other words, the fixed-bed reactor 110 may synthesize hydrocarbons containing lower olefins from raw materials containing carbon monoxide, carbon dioxide, and hydrogen.

As described above, the fixed-bed reactor 110 according to this embodiment includes a plurality of the catalyst structures 1, and the reaction tube 112 which contains the plurality of catalyst structures 1. When the plurality of catalyst structures 1 are contained in the reaction tube 112, a gas from a reaction raw material diffuses and heat conduction is improved. In addition, the fixed-bed reactor 110 can remove reaction heat more easily than a slurry reactor or a fluidized bed reactor. The reaction represented by Chemical equation (1) above is an exothermic reaction, the fixed-bed reactor 110 can efficiently produce hydrocarbons from the raw materials containing carbon dioxide. As described above, conventional catalyst pellets are used in the fixed-bed reactor 110. When the catalyst is pulverized, there is a possibility that voids in the reaction tube 112 decrease and the pressure loss increases during reaction. When a slurry reactor or a fluidized bed reactor is used, the problem of an increase in pressure loss due to pulverization does not occur. However, the catalyst structure 1 according to this embodiment can mitigate pulverization as described above. Therefore, by using the catalyst structure 1 according to this embodiment, an increase in pressure loss during reaction can be mitigated, and maintenance of catalyst exchange can be facilitated.

### [Method of Manufacturing Catalyst Structure]

Next, a method of manufacturing the catalyst structure 1 according to this embodiment will be described. The method of manufacturing the catalyst structure 1 includes a precursor preparation step, a support step, a reduction step, and an activation step.

### (Precursor Preparation Step)

The precursor preparation step is a step of preparing an iron-based catalyst precursor. The iron-based catalyst precursor may be produced, for example, by a coprecipitation method or an impregnation method, together with accessory components and a promoter, if necessary. The process for producing the iron-based catalyst precursor may include, for example, an aqueous solution preparation step, a precipitate formation step, and a first calcination step.

### (Aqueous Solution Preparation Step)

The aqueous solution preparation step is a process for preparing an aqueous solution containing iron salt and a surfactant. In the aqueous solution preparation step, an aqueous solution can be prepared by dissolving iron salt and the surfactant in water such as ion-exchange water.

Iron salt may be divalent iron salt or trivalent iron salt. Iron salt may contain at least one anion selected from the group consisting of nitrate, fluoride, chloride, bromide, iodide, phosphate, pyrophosphate and perchlorate. Iron salt may contain, for example, iron nitrate.

The surfactant may be an ionic surfactant. The surfactant may contain at least one anion selected from the group consisting of halides, sulfonate, sulfate, phosphate and carboxylate. The surfactant may contain, for example, a halide anion. The halide anion may contain a fluoride anion, a chloride anion, a bromide anion, or an iodide anion. The surfactant may contain, for example, a bromide anion. The surfactant may be a quaternary ammonium surfactant, for example, cetrimonium bromide.

The molar ratio of iron to surfactant may be from about 1:0.5 to 1:15, from about 1:0.5 to 1:12, from about 1:0.5 to 1:10, from about 1:0.5 to 1:8, from about 1:0.5 to 1:6, from about 1:0.5 to 1:4, or from about 1:0.5 to 1:2. The molar ratio of iron to surfactant may be, for example, about 1:1.

### (Precipitate Formation Step)

The precipitate formation step is a step of forming a precipitate by adding a basic salt solution to an aqueous solution produced in the aqueous solution preparation step.

Salt contained in the basic salt solution may contain at least one specific element selected from the group consisting of alkali metals, alkaline earth metals, transition metal elements of groups 3 to 7, or 9 to 11, in the periodic table, and lanthanoids. Alkali metals may contain at least one element selected from the group consisting of lithium, sodium, potassium, rubidium, and cesium. The alkaline earth metals may contain at least one element selected from the group consisting of beryllium, magnesium, calcium, strontium, and barium. The group 3 elements may contain at least one element selected from the group consisting of scandium and yttrium. Group 4 elements may contain at least one element selected from the group consisting of titanium, zirconium, and hafnium. Group 5 elements may contain at least one element selected from the group consisting of vanadium, niobium, and tantalum. Group 6 elements may contain at least one element selected from the group consisting of chromium, molybdenum and tungsten. Group 7 elements may contain at least one element selected from the group consisting of manganese, technetium and rhenium. Group 9 elements may contain at least one element selected from the group consisting of cobalt, rhodium and iridium. Group 10 elements may contain at least one element selected from the group consisting of nickel, palladium and platinum. Group 11 elements may contain at least one element selected from the group consisting of copper, silver and gold. Lanthanoids may contain at least one element selected from the group consisting of lanthanum, cerium, praseodymium, neodymium, promethium, samarium, europium, gadolinium, terbium, dysprosium, holmium, erbium, thulium, ytterbium and lutetium.

The basic salt solution may contain hydroxide anion, carbonate anion, or bicarbonate anion. Basic salt may be sodium hydroxide, lithium hydroxide, potassium hydroxide, cesium hydroxide or a combination thereof. The basic salt solution may be an aqueous solution.

The precursor preparation step may include a recovery step of recovering the precipitate by at least one of centrifugation or filtration. Further, the precursor preparation step may include a drying step of drying the recovered precipitate before a first calcination step. The drying step may be performed at a temperature of 80° C. or higher, and 120° C. or lower in the atmosphere. Cleaning of the precipitate may not be performed.

### (First Calcination Step)

The first calcination step is a step of calcinating the precipitate produced in the precipitate formation step. The first calcination step may be performed at a temperature of about 300° C. to 600° C., about 350° C. to 600° C., about 400° C. to 600° C., or about 400° C. to 550° C. The first calcination step may be performed for about 1 to 10 hours, about 1 to 8 hours, about 1 to 6 hours, about 1 to 5 hours, about 1 to 4 hours, about 1 to 3 hours, or about 1 to 2 hours.

### (Support Step)

The support step is a step of supporting the iron-based catalyst precursor with the porous carrier 3 by impregnating, drying, and calcining slurry into the porous carrier 3. The support step may include an impregnation step, a drying step, and a second calcination step.

### (Impregnation Step)

The impregnation step is a step of impregnating the porous carrier 3 with slurry. In the impregnation step, the porous carrier 3 may be immersed in the slurry, and the porous carrier 3 may be impregnated with the slurry. The porous carrier 3 may be stirred under reduced pressure while impregnating with the slurry.

The slurry contains an iron-based catalyst precursor. The slurry may contain a binder. The binder may contain at least one of an alumina binder or a silica binder. By using these binders, the iron-based catalysts 2 can be firmly bonded to each other and separation of the iron-based catalysts 2 from the porous carrier 3 can be controlled. The slurry may contain water in addition to the iron-based catalyst precursor and the binder. A weight ratio of water to the iron-based catalyst precursor in the slurry may be 0.1 to 20, 3 to 14, or 5 to 7. The weight ratio of the binder to the iron-based catalyst precursor in the slurry may be 0.1 to 2, 0.15 to 1, or 0.3 to 0.5.

The porous carrier 3 described above may be used. In order to improve adhesion of the iron-based catalyst precursor, the porous carrier 3 may be heated in the atmosphere. A heating temperature of the porous carrier 3 may be 500° C. or higher, and 700° C. or lower, or 550° C. or higher, and 650° C. or lower. A heating time of the porous carrier 3 may be, for example, 1 hour or more and 3 hours or less.

### (Drying Step)

The drying step is a step of drying the porous carrier 3 impregnated with slurry. After a liquid is sucked from the slurry impregnated into the porous carrier 3, the porous carrier 3 may be dried. The drying temperature of the porous carrier 3 may be 110° C. to 190° C., or 130° C. to 170° C.

The impregnation step and the drying step need be performed only once, or the steps may be repeated alternately. The number of repetitions may be one to seven times, two to five times, or three to four times. The support amount of the iron-based catalyst precursor to the porous carrier 3 can be increased by increasing the number of repetitions.

### (Second Calcination Step)

The second calcination step is a step of calcinating the dried porous carrier 3. The second calcinating step may be performed at a temperature of about 300° C. to 600° C., about 350° C. to 600° C., about 400° C. to 600° C., about 400° C. to 550° C., or about 400° C. to 550° C. The second calcinating step may be performed for about 1 to 10 hours, about 1 to 8 hours, about 1 to 6 hours, about 1 to 5 hours, about 1 to 4 hours, about 1 to 3 hours, or about 1 to 2 hours.

### (Reduction Step)

The reduction step is a step of reducing the iron-based catalyst precursor supported with the porous carrier 3. In the reduction step, the iron-based catalyst precursor reacts with a reducing gas. The reducing gas is a gas capable of reducing iron oxide, for example, a hydrogen gas or a carbon monoxide gas. The reaction temperature may be 200° C. to 500° C., or 300° C. to 400° C. The reaction time may be, for example, 1 to 12 hours, 3 to 10 hours, or 5 to 8 hours.

### (Activation Step)

The activation step is a step of activating a reduced iron-based catalyst precursor with an activation gas to produce an iron-based catalyst 2 containing an Fe₅C₂ phase and at least one of an Fe₂O₃ phase or an Fe₃O₄ phase. The activation gas contains at least one of carbon monoxide or carbon dioxide. The activation gas may be, for example, a mixture gas containing hydrogen and at least one of carbon monoxide or carbon dioxide. The activation pressure may be 0.7 MPa to 1.1 MPa or 0.8 MPa to 1 MPa. The activation temperature may be 250° C. to 370° C., or 300° C. to 350° C. The activation time may be, for example, 1 hour or more, 2 hours or more, 3 hours or more, or 5 hours or more. The activation time may be, for example, 100 hours or less, 50 hours or less, 25 hours or less, 10 hours or less, or 5 hours or less.

According to the steps above, the catalyst structure 1 including the iron-based catalyst 2 and the porous carrier 3 supporting the iron-based catalyst 2 can be produced to synthesize hydrocarbons containing lower olefins.

As described above, the method of manufacturing the catalyst structure 1 according to the present embodiment includes a support step, a reduction step, and an activation step. In the support step, the porous carrier 3 is impregnated with a slurry containing a binder and the iron-based catalyst precursor, dried, and calcined to support the iron-based catalyst precursor. In the reduction step, the iron-based catalyst precursor supported with the porous carrier 3 is reduced. In the activation step, the reduced iron-based catalyst precursor is activated with an activation gas containing at least one of carbon monoxide or carbon dioxide to produce the iron-based catalyst 2 containing an Fe₅C₂ phase, and at least one of an Fe₂O₃ phase or an Fe₃O₄ phase. The catalyst structure 1 includes an iron-based catalyst 2 and a porous carrier 3 supporting the iron-based catalyst 2, and synthesizes hydrocarbons containing lower olefins.

With the method of manufacturing the catalyst structure 1 according to this embodiment, as described above, the catalyst structure 1 capable of mitigating pulverization can be produced.

### EXAMPLE

Hereinafter, this embodiment will be described in more detail with reference to examples and comparative examples, but this embodiment is not limited to these examples.

### Example 1

First, a catalyst structure according to this example was prepared using a prepared iron-based catalyst precursor and a porous carrier as follows.

### (Iron-Based Catalyst Precursor)

Iron salt of iron nitrate (Fe(NO₃)₂·9H₂O) and a surfactant were dissolved in ion-exchange water to prepare a homogeneous aqueous solution, to which an aqueous solution of alkali metal salt as an aqueous NaOH solution was added dropwise to form a precipitate containing iron salt and alkali metal. After aging at room temperature, the precipitate was filtered for solid-liquid separation, and the solid was recovered. The precipitate was not washed, and the recovered solid was dried at 100° C. and calcined at 450° C. in air. After calcining, the oxide was pulverized in a mortar to obtain test samples of iron-based catalyst precursors.

### (Porous Carrier)

A cubic NiFeCr alloy 3 mm in length, 3 mm in width, and 3 mm in height was used as a carrier. The cell size (pore size) of the carrier was 800 μm and the porosity was 90% to 95%. In order to improve the adhesion of iron catalyst powder, the porous carrier was heated up to 600° C. at 10° C./min in air, heated at 600° C. for 2 hours, and cooled in a furnace to obtain a pretreated porous carrier.

### (Support Step)

Next, the iron-based catalyst precursor, purified water, and an alumina binder were mixed in a container at a weight ratio of iron-based catalyst precursor: purified water: alumina binder=1:6.2:0.32, to prepare a slurry. The pretreated porous carrier was immersed in the prepared slurry, and the slurry was reduced in pressure while stirring in a vacuum defoaming device with a stirrer. After stirring the slurry, the liquid was suctioned using a suction dryer, and the porous carrier was dried at 150° C. Impregnation and drying were repeated three times in total. The weight of the iron-based catalyst precursor to the support carrier (iron-based catalyst precursor support amount) was measured to be 26.4% by weight. It should be noted that the iron-based catalyst precursor support amount was calculated by subtracting the alumina support amount from a material supported with the porous carrier. The catalyst precursor support carrier thus obtained was heated at 5° C./min in air and calcined at 450° C. for 2 hours.

### (Reaction System)

Next, in order to reduce and activate the calcined catalyst precursor carrier, a reaction system 100 with the fixed-bed reactor 110 was prepared as illustrated in FIG. 3. Specifically, a calcined catalyst precursor carrier 111 prepared as described above was filled into the reaction tube 112 having an outer diameter of ¼ inch, and the reaction tube 112 was installed in the electric furnace 113. A hydrogen tank 121, a nitrogen tank 122, and an activated mixed gas tank 123 were prepared as feed gases to the reaction tube 112. The supply of hydrogen gas from the hydrogen tank 121 to the reaction tube 112 was controlled by a solenoid valve 124 and a mass flow controller (MFC) 125. The supply of nitrogen gas from the nitrogen tank 122 to the reaction tube 112 was controlled by an MFC 126. The supply of activated mixed gas from the activated mixed gas tank 123 to the reaction tube 112 was controlled by a high-pressure MFC 127. These gases were mixed by a mixer 128 as necessary.

An inlet side back pressure valve 131 and a safety valve 132 were installed at an inlet side of the reaction tube 112, and an outlet side back pressure valve 133 was installed at an outlet side of the reaction tube 112. An inlet side temperature of the catalyst precursor carrier 111 was measured with a thermocouple inlet side thermometer 141, and an outlet side temperature of the catalyst precursor carrier 111 was measured with a thermocouple outlet side thermometer 142. Further, an inlet side pressure of the reaction tube 112 was measured with an inlet side pressure gauge 143, and an outlet side pressure of the reaction tube 112 was measured with an outlet side pressure gauge 144. When the pressure loss increased, the openings of the inlet side back pressure valve 131 and the outlet side back pressure valve 133 were adjusted so that the pressure of the inlet side pressure gauge 143 became constant and the pressure of the outlet side pressure gauge 144 decreased.

Next, the catalyst precursor was reduced and activated under the test conditions shown in Table 1 as follows.

TABLE 1

Test Conditions

| Sample | Reduction | Activation |
|---|---|---|
| Name | Time (hours) | Time (hours) |
| Run 0 | 0 | 0 |
| Run 1 | 6 | 0 |
| Run 2 | 6 | 3 |
| Run 3 | 6 | 6 |
| Run 4 | 6 | 24 |

### (Reduction)

Nitrogen gas was supplied to the reaction tube 112 at 28 NmL/min. Next, the reaction tube 112 was heated, and the temperature was raised from room temperature to 400° C. After the temperature of the reaction tube 112 reached 400° C., the feed gas was switched from nitrogen to hydrogen (28 NmL/min), and reduction was started. 6 hours after starting the supply of hydrogen, the feed gas to the reaction tube 112 was switched from hydrogen to nitrogen. The heating to the reaction tube 112 was stopped, and the reaction tube 112 was cooled naturally.

### (Activation)

The supply of nitrogen gas to the reaction tube 112 was started. Next, the reaction tube 112 was pressurized to about 0.65 MPaG. Next, the temperature of the reaction tube 112 was raised from room temperature to 325° C. at 10° C./min. The supply of nitrogen gas to the reaction tube 112 was stopped, and the reaction tube 112 was pressurized to 0.9 MPaG by the activation gas at 28 NmL/min. After the time indicated in Table 1 had elapsed, the supply of the activation gas was stopped. The pressure was lowered to normal pressure while the reaction tube 112 was cooled naturally. The gas was switched to O₂-0.5%/N₂ gas, and the catalyst was removed after surface oxidation for more than 1 hour.

### Comparative Example 1

Reduction and activation were performed in the same manner as in Example 1, except that the test sample prepared in the same manner as in Example 1 was extruded to prepare pellets with a diameter of 3 mm, and the pellets were filled in the reaction tube 112.

### Comparative Example 2

Reduction and activation were performed in the same manner as in Example 1, except that the calcination time of the test sample was changed from 450° C. to 550° C.

### [Evaluation]

The pulverization state, pressure loss, and XRD of the catalyst structures after the activation treatment were evaluated.

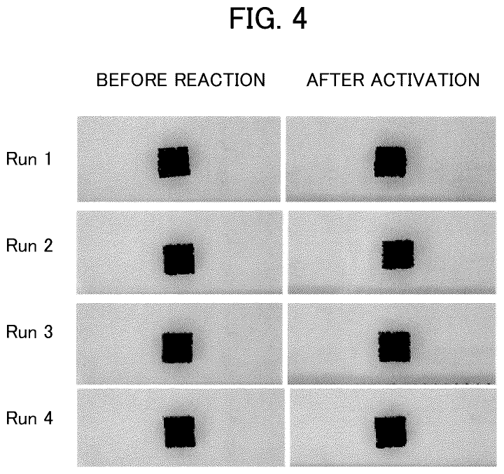

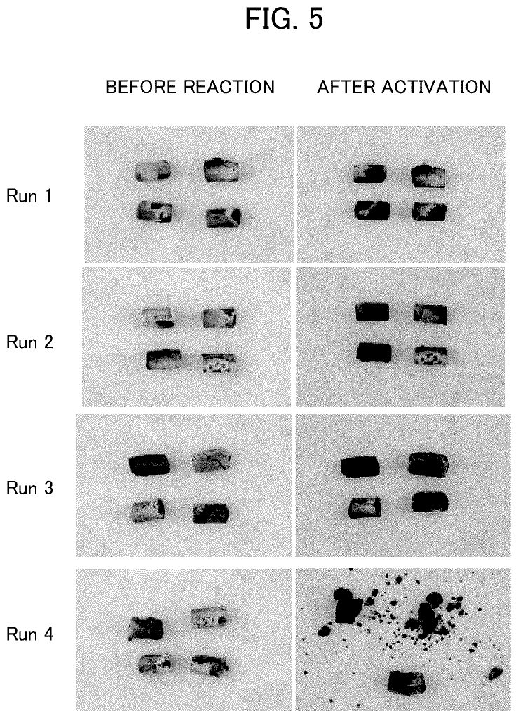

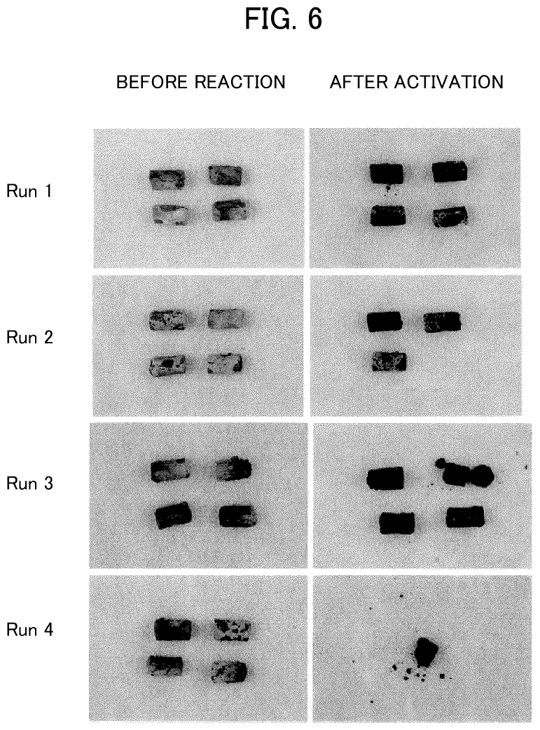

FIGS. 4 to 6 are photographs showing the appearance of the catalyst structures of Example 1, Comparative Example 1, and Comparative Example 2 before reaction and after activation in Run 1 to Run 4. As shown in FIGS. 4 to 6, no pulverization was observed in the catalyst structure of Example 1 even after activation in Run 4. However, pulverization was observed in the catalyst structure of Comparative Example 1 after activation in at least Run 4. Further, pulverization was also observed in the catalyst structure of Comparative Example 2, which was considered to be made stronger by setting the calcination temperature higher than that of Comparative Example 1, after activation in at least Run 4.

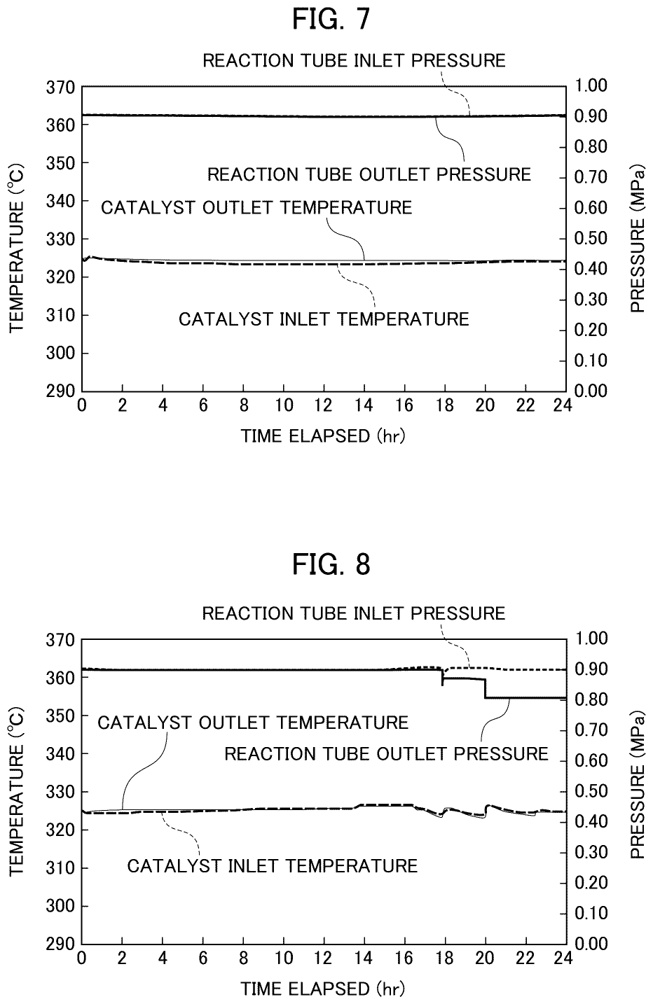

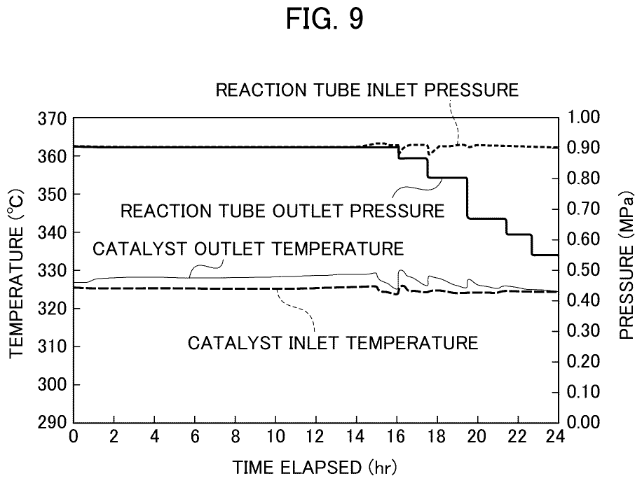

FIGS. 7 to 9 are graphs illustrating the relationship between activation time and pressure loss when the catalyst structures of Example 1, Comparative Example 1, and Comparative Example 2 were reacted under the conditions of Run 4. As illustrated in FIG. 7, in the catalyst structure of Example 1, a reaction tube inlet pressure and a reaction tube outlet pressure remained constant until activation in Run 4 was completed, and no pressure loss was confirmed. However, as illustrated in FIGS. 8 and 9, in the catalyst structures of Comparative Example 1 and Comparative Example 2, the reaction tube outlet pressure continued to decrease relative to the reaction tube inlet pressure during activation in Run 4, and it was confirmed that pressure loss occurred.

From the results of FIGS. 4 to 9, it can be seen that the catalyst structure of Example 1 can mitigate pulverization and the pressure loss as compared with the catalyst pellets formed by extrusion molding.

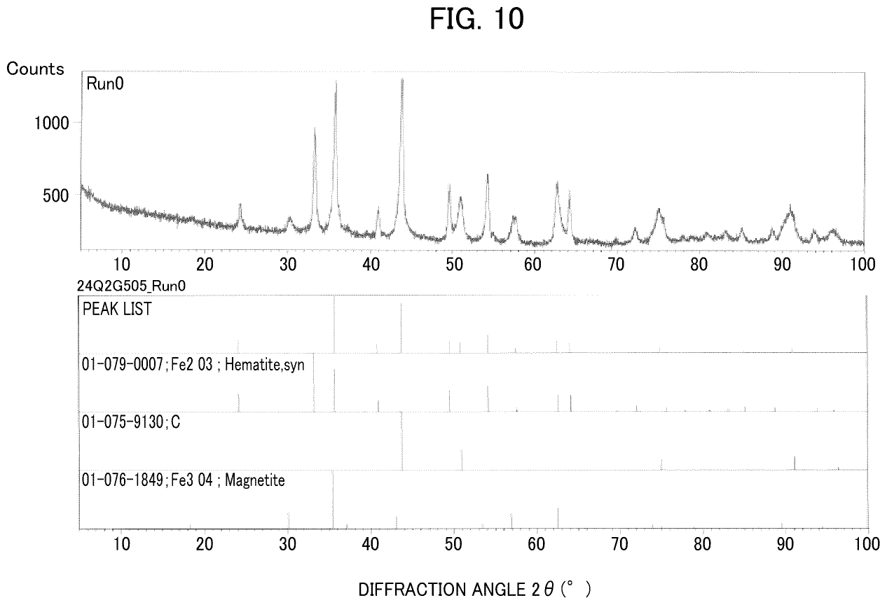

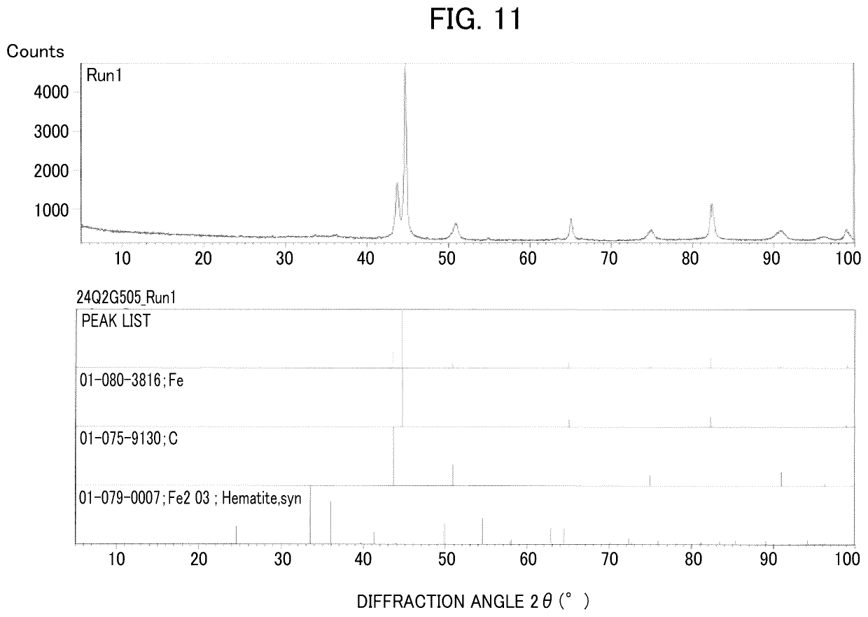

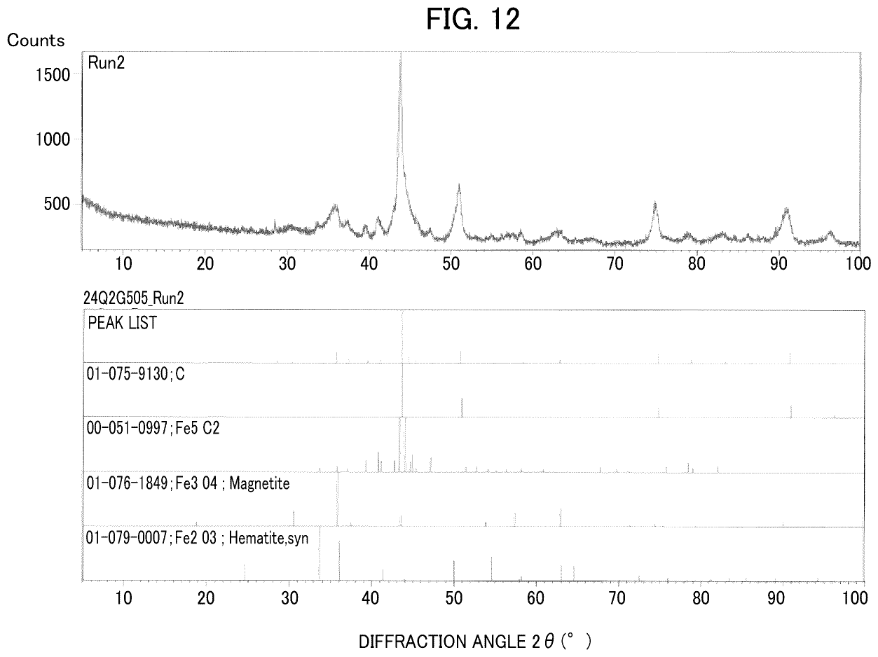

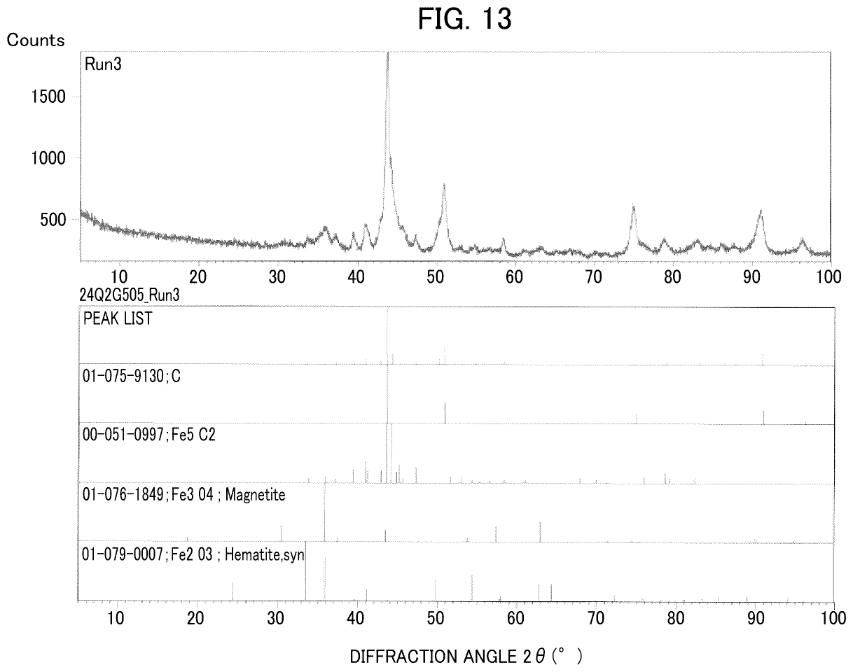

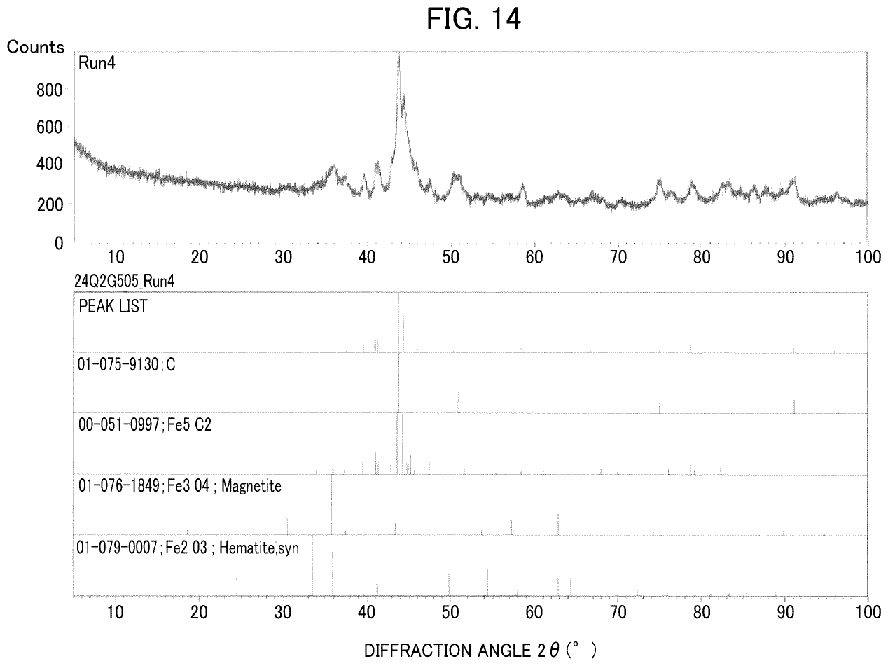

FIGS. 10 to 14 illustrate X-ray diffraction patterns of an iron-based catalyst after the reaction of the catalyst structure of Example 1 under the conditions of Run 0, Run 1, Run 2, Run 3, and Run 4, respectively. As illustrated in FIG. 10, a main component of the iron-based catalyst after calcination was iron oxide. As illustrated in FIG. 11, a main component of the iron-based catalyst after reduction was iron. As illustrated in FIGS. 12 to 14, the iron-based catalyst after activation contained the Fe₅C₂ phase, and at least one of Fe₂O₃ phase or Fe₃O₄ phase. However, carbon was also produced.

The iron-based catalyst according to Example 1 contains at least one of the Fe₂O₃ phase or the Fe₃O₄ phase, so that the reverse shift reaction as shown in the Chemical equation (2) above can progress, and that carbon monoxide can be produced from a raw material containing carbon dioxide and hydrogen. The iron-based catalyst according to Example 1 contains the Fe₅C₂ phase, so that the FT reaction as shown in the Chemical equation (1) above can progress, and that hydrocarbons containing lower olefins can be produced from raw materials containing carbon monoxide and hydrogen.

Although several embodiments have been described, the embodiments may be modified or varied based on the disclosure above. All components of the embodiments above and all features described in the claims may be individually extracted and combined as long as they are consistent with each other.

This disclosure may, for example, contribute to Goal 13 of the United Nations-led Sustainable Development Goals (SDGs): Take urgent action to combat climate change and its impacts.

What is claimed is:

1. A catalyst structure comprising:
an iron-based catalyst containing an Fe₅C₂ phase, and at least one of an Fe₂O₃ phase or an Fe₃O₄ phase; and
a porous carrier for supporting the iron-based catalyst, wherein
the catalyst structure configures to synthesize hydrocarbons containing lower olefins.

2. The catalyst structure according to claim 1, wherein a volume of the catalyst structure is 0.5 mm³ or more, and 1000 mm³ or less.

3. The catalyst structure according to claim 1, wherein the porous carrier is formed of metal.

4. The catalyst structure according to claim 1, wherein porosity of the porous carrier is 85% to 95%.

5. A fixed-bed reactor comprising:
a plurality of catalyst structures according to claim 1; and
a reaction tube which contains the plurality of catalyst structures.

6. The fixed-bed reactor according to claim 5, wherein the hydrocarbons are synthesized from a raw material containing carbon dioxide and hydrogen.

7. The fixed-bed reactor according to claim 5, wherein the hydrocarbons are synthesized from a raw material containing carbon monoxide, carbon dioxide, and hydrogen.

8. A method of manufacturing a catalyst structure comprising:
supporting an iron-based catalyst precursor with a porous carrier by impregnating a slurry containing a binder and the iron-based catalyst precursor into a porous carrier, and drying and calcining the slurry;
reducing the iron-based catalyst precursor supported with the porous carrier; and
activating the reduced iron-based catalyst precursor with an activation gas containing at least one of carbon monoxide or carbon dioxide to produce an iron-based catalyst containing an Fe₅C₂ phase, and at least one of an Fe₂O₃ phase or an Fe₃O₄ phase, wherein
the catalyst structure comprises the iron-based catalyst and the porous carrier for supporting the iron-based catalyst, and configures to synthesize hydrocarbons containing lower olefins.

9. The method of manufacturing a catalyst structure according to claim 8, wherein the binder comprises at least one of an alumina binder or a silica binder.
